(19) United States
(12) Plant Patent Application Publication
(10) Pub. No.: US 2011/0191924 P1
(43) Pub. Date: Aug. 4, 2011

(54) SPATHIPHYLLUM PLANT NAMED 'SWEET ROMANO'

(50) ;

(76) Inventor: Petrus Olsthoorn, Honselersdijk (NL)

(21) Appl. No.: 12/658,361

(22) Filed: Feb. 4, 2010

(51) Int. Cl. A01H 5/00

(52) U.S. Cl. PLT/364

(57) ABSTRACT

A new and distinct Spathiphyllum cultivar named ‘Sweet Romano’ is disclosed, characterized by glossy, dark green foliage. The new cultivar is a Spathiphyllum, typically suited for ornamental container use.

### LATIN NAME OF THE GENUS AND SPECIES

Spathiphyllum hybrid

### VARIETY DENOMINATION

‘Sweet Romano’

### BACKGROUND OF THE INVENTION

The new variety originated from a planned crossing of the seed parent, an unpatented proprietary variety referred to as ‘98286-29’ with the pollen parent, the unpatented, proprietary variety referred to as ‘95338-125’. The new variety was discovered by the inventor Petrus Olsthoorn in 2004 at a commercial greenhouse in Honselersdijk, the Netherlands.

After selecting and isolating the new cultivar, asexual reproduction of the new cultivar ‘Sweet Romano’ was first performed in a commercial laboratory in Honselersdijk, the Netherlands by tissue culture in December of 2004. ‘Sweet Romano’ has since produced several generations and has shown that the unique features of this cultivar are stable and reproduced true to type.

### SUMMARY OF THE INVENTION

The cultivar ‘Sweet Romano’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘Sweet Romano.’ These characteristics in combination distinguish ‘Sweet Romano’ as a new and distinct Spathiphyllum cultivar:

1. Very glossy foliage.

2. Consistently dark green foliage.

### PARENTAL COMPARISON

Plants of the new cultivar ‘Sweet Romano’ are similar to both parent varieties in most horticultural characteristics. However, ‘Sweet Romano’ differs in producing wider diameter foliage than either parent. Additionally, ‘Sweet Romano’ produces glossier foliage than either parent variety.

### COMMERCIAL COMPARISON

Plants of the new cultivar ‘Sweet Romano’ are best compared to the commercial variety ‘Sweet Benito’ U.S. Plant Pat. No. 13,960. Plants of ‘Sweet Romano’ are similar to plants of ‘Sweet Benito’ in most horticultural characteristics, however, plants of ‘Sweet Romano’ produce darker green, glossier leaves than ‘Sweet Benito.’

### BRIEF DESCRIPTION OF THE PHOTOGRAPH

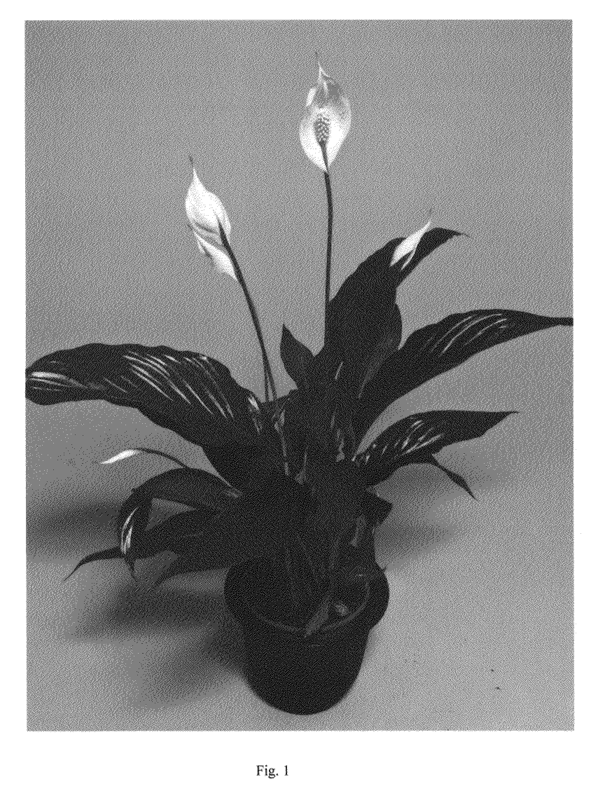

The accompanying photograph in FIG. 1 illustrates in full color a typical plant of ‘Sweet Romano’ grown in a greenhouse in Honselersdijk, the Netherlands. This plant is approximately 10 months old, shown in a 14 cm pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart except where general terms of ordinary dictionary significance are used. The following observations and measurements describe ‘Sweet Romano’ plants grown in a greenhouse in Honselersdijk, the Netherlands. Temperatures ranged from 18° C. to 20° C. at night to 19° C. to 20° C. during the day. No artificial light, photoperiodic treatments or chemical treatments were given to the plants. Measurements and numerical values represent averages of typical plant types.
Botanical classification: Spathiphyllum hybrid ‘Sweet Romano’
Age of the plant described: Approximately 10 months
Container size of the plant described: 14 cm commercial container.
Propagation:
Time to initiate roots: Approximately 7-14 days from an initial tissue culture plantlet at approximately 22° C.
Time to produce rooted plantlet: Approximately 4 weeks at approximately 20 to 22° C.
Root description: Moderately fibrous, not fleshy, moderately dense, colored near RHS Yellow-White 158D.
Plant:
Growth habit: Clump-forming, upright
Plant shape: Broad inverted triangle with flowers above leaf plane
Height: Approximately 39.8 cm to top of highest leaf, Approximately 61.0 cm to top of highest spathe.
Plant spread: Approximately 51.0 cm in a 14 cm
Growth rate: Low to moderate
Branching characteristics: No branching. Leaves emerge direct from base of plant.
Number of clumps of leaves: Average 8
Number of leaves per clump: Average 4
Number of leaves per plant: Approximately 32
Foliage:
Leaf:
   Arrangement.—alternate, growing directly from the soil.
   Average length (excluding petiole).—Approximately 25.3 cm.
   Average width.—Approximately 7.7 cm.
   Shape of blade.—narrow ovate to lanceolate.
   Aspect.—Slightly carinate, slightly curved, pointed outward-upright.
   Apex.—long apiculate.
   Base.—attenuate.
   Margin.—entire, moderately undulate.
   Texture of top surface.—smooth, highly glossy.
   Texture of bottom surface.—smooth, moderately glossy.
   Color.—Young foliage upper side: Near R.H.S. Green 141A Young foliage under side: Near R.H.S. Green 137B Mature foliage upper side: Near R.H.S. Green 137B Mature foliage under side: Near R.H.S. Green 137B.
Venation:
   Type.—pinnate.
   Venation coloration upper side.—Near R.H.S. Green N137A.
   Venation coloration under side.—Near R.H.S. Yellow-Green 145A.
Petiole (excluding geniculum):
   Length.—Approximately 17.3 cm.
   Width.—At geniculum: Approximately 0.3 cm Above clump: Approximately 0.6 cm.
   Color.—Near RHS Green 137C.
   Strength.—moderately strong.
Geniculum:
   Length.—Approximately 2.9 cm.
   Width.—Approximately 0.4 cm.
   Color.—Near RHS Green 137D.
Inflorescence:
Arrangement: Inflorescences are carried slightly above the leaf plane
Flowering habit: Continuous
Quantity of spathes per plant: On average 4 fully open spathes in good condition at one time.
Natural flowering season: Late summer to midwinter
Time to flowering: Approximately 40 weeks from a rooted young plant.
Fragrance: Moderately strong, sweet and pleasant (typical Spathiphyllum scent)
Self-cleaning or persistent: Persistent
Flower longevity: Flowers stay in good condition approximately 3 weeks on the plant.
Spathe:
   Aspect.—upright.
   Length.—Approximately 11.2 cm.
   Width.—Approximately 4.9 cm.
   Shape.—ovate, concave.
   Margin.—entire.
   Apex.—apiculate to aristate.
   Base.—attenuate.
   Color.—Front when opening: Near RHS White 155C Back when opening: Near RHS White 155C Front when mature: Near RHS White N155A Back when mature: Near RHS White N155A Fading to: not fading.
Spadix:
   Shape.—oblong.
   Tip.—obtuse.
   Base.—obtuse.
   Length.—Approximately 3.0 cm.
   Width.—Approximately 1.25 cm.
   Color.—When opening: Near RHS White 155D Mature: Near in between RHS Orange-White 159D.
   Quantity of flowers per spadix.—Approximately 90.
   Spadix flower arrangement.—spirally placed on spadix.
   Spadix flower diameter.—Approximately 0.35 cm.
   Spadix flower depth.—Approximately 0.25 cm.
Peduncle:
   Length.—Approximately 41.4 cm.
   Width.—Approximately 0.35 cm.
   Color.—Near RHS Yellow-Green 144A.
   Strength.—moderately strong.
Reproductive organs:
General description of visible parts: Three stigmas are placed on top of each ovary (length of ovary approximately 0.35 cm), stigma diameter approximately 0.1 cm, colored Near RHS White N155D.
Other characteristics:
Seeds and fruits: No seeds/fruits observed.
Disease/pest resistance: Neither resistance nor susceptibility to the normal pests and diseases of Spathiphyllum has been observed.
Temperature tolerance: Indoor plant, no frost tolerance

What is claimed is:

1. A new and distinct cultivar of Spathiphyllum hybrid plant named ‘Sweet Romano’ as herein illustrated and described.
